(19) United States
(12) Patent Application Publication
(10) Pub. No.: US 2023/0358730 A1
(43) Pub. Date: Nov. 9, 2023

(54) SIMULATED SYNOVIAL FLUIDS FOR MODELING DEGENERATIVE JOINT DISEASES AND SCREENING FOR THERAPEUTICS FOR SAME

(71) Applicants: Georgia Tech Research Corporation, Atlanta, GA (US); Emory University, Atlanta, GA (US)

(72) Inventors: Annie Bowles-Welch, Atlanta, GA (US); Nick Willett, Atlanta, GA (US); Andres J. Garcia, Atlanta, GA (US); Linda Kippner, Atlanta, GA (US); Krishnendu Roy, Atlanta, GA (US); Rebecca S. Schneider, Atlanta, GA (US); Hazel Stevens, Atlanta, GA (US); Carolyn Yeago, Atlanta, GA (US)

(21) Appl. No.: 18/312,321

(22) Filed: May 4, 2023

Related U.S. Application Data
(60) Provisional application No. 63/364,153, filed on May 4, 2022.

(51) Int. Cl. G01N 33/50

(52) U.S. Cl. CPC G01N 33/5091; G01N 2333/475; G01N 2333/54; G01N 2333/555; G01N 2800/105

(57) ABSTRACT

An exemplary embodiment of the present disclosure provides a composition comprising two or more cytokines, and one or more of keratan sulfate, chondroitin sulfate, or hyaluronic acid. The composition can simulate a fluid from a patient. The simulated fluid has a viscosity, a storage modulus, and a loss modulus similar to that of patient-derived synovial fluid. A method for making a composition for simulating a fluid from a patient is also disclosed. The method includes creating a mixture comprising two or more cytokines, and one or more of keratan sulfate, chondroitin sulfate, or human serum albumin. The method also includes adding a low molecular weight hyaluronic acid to the mixture, adding a high molecular weight hyaluronic acid to the mixture, and incubating the mixture for a predetermined time at a temperature ranging from approximately 0° C. to approximately 10° C.

### CROSS-REFERENCE TO RELATED APPLICATIONS

This application claims the benefit of U.S. Provisional Application No. 63/364,153, filed on 4 May 2022, which is incorporated herein by reference in its entirety as if fully set forth below.

### FEDERALLY SPONSORED RESEARCH STATEMENT

This invention was made with government support under Agreement No. 75F40120C00207, awarded by United States Food and Drug Administration. The government has certain rights in the invention.

### FIELD OF THE DISCLOSURE

The various embodiments of the present disclosure relate generally to simulated fluids for screening cell therapies, and more specifically to simulated synovial fluid for osteoarthritis research and screening.

### BACKGROUND

Knee osteoarthritis is one of the most common degenerative joint diseases and yet the various risk factors, e.g. injury, obesity, genetics, render the severity and progression difficult to elucidate. Consequently, effective treatments for osteoarthritis, and knee osteoarthritis are limited. Cell therapies are evaluated as non-surgical alternatives for osteoarthritis, although efficacy outcomes are confounded by the patient-specific osteoarthritis environment that therapeutic cells respond to following intra-articular injection.

The osteoarthritis environment can be evaluated from a patient’s synovial fluid, which is the viscous fluid between articular cartilages of synovial joints that provides lubrication and reduces friction during movement. Cellular responses can be attributed to the components found in the patient’s synovial fluid, which are highly variable among various osteoarthritic conditions. Thus, understanding how cell therapies respond to osteoarthritis synovial fluid may provide valuable insight into potential mechanisms of action and efficacy outcomes. Thus, there is a need for a developed simulated synovial fluid (SF) product that may facilitate defined and reproducible predictive outcomes of therapeutic efficacies of cell therapies and ultimately advance a promising treatment for osteoarthritis.

### BRIEF SUMMARY

An exemplary embodiment of the present disclosure provides a simulated synovial fluid comprising two or more cytokines, and one or more of keratan sulfate, chondroitin sulfate, or hyaluronic acid. The composition can simulate a fluid from a patient.

In some embodiments, at a shear rate of approximately 0.1 reciprocal seconds (s-1), the composition can include a viscosity ranging from approximately 100 millipascal second (mPa·s) to approximately 10,000 mPa·s.

In some embodiments, at an angular frequency of approximately 0.1 radians per second (rad/sec), the composition can include a loss modulus ranging from approximately 0.01 pascal (Pa) to approximately 1 Pa.

In some embodiments, at an angular frequency of approximately 0.1 radians per second (rad/sec), the composition can include a storage modulus ranging from approximately 0.001 pascal (Pa) to approximately 1.5 Pa.

In some embodiments, the cytokines can include one or more of basic fibroblast growth factor (bFGF), epidermal growth factor (EGF), eotaxin, granulocyte-colony stimulating factor (G-CSF), granulocyte-macrophage colony-stimulating factor (GM-CSF), hepatocyte growth factor (HGF), IFN-alpha, IFN-gamma, IL-2, IL-2R, IL-6, IL-7, IL-8, IL-12, IL-13, IL-15, IL-17A, interferon gamma-induced protein 10 (IP-10), monocyte chemoattractant protein-1 (MCP-1), monokine induced by interferon-gamma (MIG), macrophage inflammatory protein-1 alpha (MIP-1alpha), macrophage inflammatory protein-1 beta (MIP-1beta), regulated on activation-normal T cell expressed and secreted (RANTES), tumor necrosis factor alpha (TNF-alpha), or vascular endothelial growth factor (VEGF).

In some embodiments, the cytokines can include bFGF, G-CSF, HGF, MIG, and MCP-1. In some embodiments, the bFGF can include at least 40% based on a total weight per volume of cytokines in the composition. The bFGF can include from approximately 50 wt.% to approximately 60 wt.% based on the total weight per volume of the cytokines in the composition.

In some embodiments, the composition can further comprise human serum albumin (HSA).

In some embodiments, the composition can further comprise both a high molecular weight hyaluronic acid and a low molecular weight hyaluronic acid.

In some embodiments, the composition can include high molecular weight hyaluronic acid present from approximately 5% to approximately 20%, based on a total weight per volume of the composition. The composition can include low molecular weight hyaluronic acid present from approximately 0.01% to approximately 5%, based on a total weight per volume of the composition.

In some embodiments, the high molecular weight hyaluronic acid can be present in a higher concentration within the composition compared to the low molecular weight hyaluronic acid.

In some embodiments, the simulated fluid can have a viscosity similar to a viscosity of patient-derived synovial fluid.

In some embodiments, the simulated fluid can have a storage moduli similar to a storage moduli of patient-derived synovial fluid.

In some embodiments, the simulated fluid can have a loss moduli similar to a loss moduli of patient-derived synovial fluid.

In some embodiments, the patient can comprise a degenerative joint disease.

Another aspect of the present disclosure provides a method of making a composition for simulating a fluid from a patient. The method can include creating a mixture and incubating the mixture for a predetermined time at a temperature ranging from approximately 0° C. to approximately 10° C. The mixture can include two or more cytokines and one or more of keratan sulfate, chondroitin sulfate, or human serum albumin. The method can include adding a low molecular weight hyaluronic acid to the mixture and adding a high molecular weight hyaluronic acid to the mixture.

In some embodiments, the method can further include obtaining one or more samples of patient-derived synovial fluid from one or more patients and analyzing a protein content of the one or more samples of patient-derived synovial fluid.

In some embodiments, the predetermined time can range from approximately 2 hours to approximately 72 hours.

Another aspect of the present disclosure provides a method of analyzing one or more cellular responses from human cells. The method can include exposing the sample of human cells to a simulated synovial fluid comprising the composition described herein, and performing a 2D synovial fluid potency assay on the human cells exposed to the simulated synovial fluid.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides a graph showing concentrations of proteins in patient-derived synovial fluid, in accordance with an exemplary embodiment of the present invention.

FIG. 1B provides a heatmap characterization of osteoarthritis patient-derived synovial fluid characterization analyzed by Z-score on the left axis and total protein on the right axis, in accordance with an exemplary embodiment of the present invention.

FIG. 2A provides a plot of total protein concentration based on KL score in de-identified osteoarthritis patient-derived synovial fluid, including cytokines, chemokines, and growth factors, in accordance with an exemplary embodiment of the present invention.

FIGS. 2B and 2C provide graph showing sulfated glycosaminoglycan concentration based on KL score in patient-derived synovial fluid, including chondroitin sulfate (FIG. 2B) and keratan sulfate (FIG. 2C), in accordance with an exemplary embodiment of the present invention.

FIG. 3A is an electrophoresis analysis comparison of intact hyaluronic acid and degraded hyaluronic acid for various osteoarthritis patient-derived synovial fluids compared to non-osteoarthritis patient synovial fluid controls, in accordance with an exemplary embodiment of the present invention.

FIG. 3B provides a graph showing categorized degradation of hyaluronic acid for various osteoarthritis patient-derived synovial fluids compared to non-osteoarthritis patient synovial fluid controls, in accordance with an exemplary embodiment of the present invention.

FIGS. 4A through 4C provide viscoelastic properties of patient-derived synovial fluid, including viscosity (FIG. 4A), loss modulus (FIG. 4B), and storage modulus (FIG. 4C), in accordance with an exemplary embodiment of the present invention.

FIGS. 4D through 4F provide compare viscoelastic properties of patient-derived synovial fluid to that of example simulated synovial fluid (simSF) product, including viscosity (FIG. 4D), loss modulus (FIG. 4E), and storage modulus (FIG. 4F), in accordance with an exemplary embodiment of the present invention.

FIG. 5 provides a principal component analysis (PCA) of patient-derived synovial fluid, in accordance with an exemplary embodiment of the present invention.

FIG. 6 provides a principal component analysis (PCA) comparison of response of mammalian cells to patient-derived synovial fluid compared to example simulated synovial fluid using 2D synovial fluid exposure potency assay, in accordance with an exemplary embodiment of the present invention.

FIG. 7 provides a constellation plot showing a relationship of secretory responses from bone marrow-derived MSCs using a 3D microfluidic synovial fluid exposure potency assay, in accordance with an exemplary embodiment of the present invention.

FIG. 8 provides a schematic illustration of a method of manufacturing an example simSF product, in accordance with an exemplary embodiment of the present invention.

FIGS. 9A through 9C provide schematic illustrations of conducting an in vitro assay with MSCs, macrophages, or PBMCs with an example simSF product, in accordance with an exemplary embodiment of the present invention.

FIG. 10 is a flowchart of a method of manufacturing an example simSF product, in accordance with the disclosed technology.

FIG. 11 is a flowchart of a method of analyzing one or more cellular responses from mammalian cells using an example simulated synovial fluid, in accordance with the disclosed technology.

### DETAILED DESCRIPTION

To facilitate an understanding of the principles and features of the present disclosure, various illustrative embodiments are explained below. The components, steps, and materials described as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

In the claims appended hereto, the term “a” or “an” is intended to mean “one or more,” and the term “comprise” and variations thereof such as “comprises” and “comprising,” when preceding the recitation of a step or an element, are intended to mean that the addition of further steps or elements is optional and not excluded.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%.

The term “sample” refers to any mixture comprising a cell, e.g., tissue. Biological samples may be obtained from animals (including humans) and encompass fluids, solids, tissues, and gases. Biological samples include blood products, such as plasma, serum and the like.

“Osteoarthritis” refers to any joint disease that results from a breakdown of joint cartilage and underlying bone. The symptoms of osteoarthritis can progress slowly (e.g., over months or years), or may occur as a result of an injury and include joint swelling, decreased range of motion, weakness, or numbness of the infected area. The most commonly involved joints include the knee, the fingers, the joint at the base of the thumb, hip joints, and joints of the neck and lower back. Causes of osteoarthritic symptoms can include prior joint injury, abnormal joint or limb development, or inherited factors.

The term “KL score” or “KL classification” refers to the Kellgren-Lawrence classification of osteoarthritis, which is a five-grade classification scheme based on plain radiographs of eight joints including the distal interphalangeal joint (DIP), metacarpophalangeal joint (MCP), first carpometacarpal joint (CMC), wrist, cervical spine, lumbar spine, hips, and knees. Grade 0 is defined as no radiographic features of osteoarthritis; grade 1 is defined as “doubtful” classification and presents radiographic features of minute osteophyte and possible osteophytic lipping; grade 2 is defined as “mild” classification and presents radiographic features of definite osteophyte and normal joint space; grade 3 is defined as “moderate” classification and presents radiographic features of moderate joint space reduction; and grade 4 is defined as “severe” classification and presents radiographic features of marked joint space greatly reduced and subchondral sclerosis. The KL classification may also assist healthcare providers with a treatment algorithm to guide clinical decision-making, specifically defining which patients may benefit most from surgical management.

The term “cytokine” refers to a type of protein that is released from certain immune and non-immune cells that have an effect on the immune system. Some cytokines can stimulate the immune system and other cytokines can slow it down. Example cytokines can include, without limitation, interleukins (IL), interferons, or colony-stimulating factors.

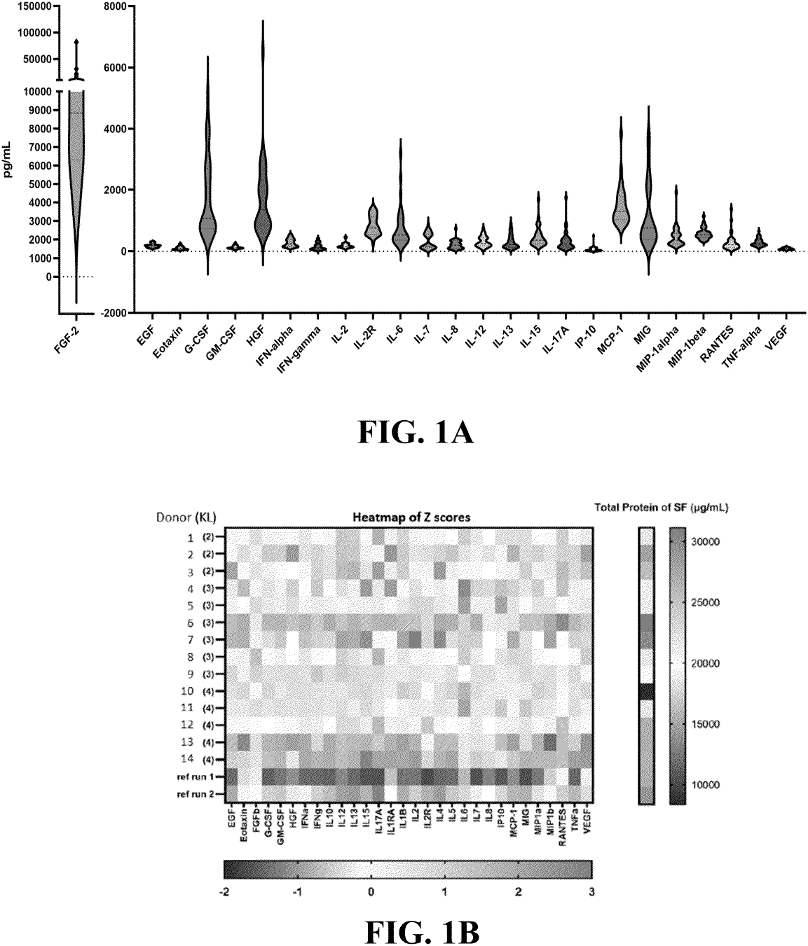

In joints where osteoarthritis has developed, the cells in and around the joint release inflammatory substances into the synovial fluid of the joint. Thus, the synovial fluid carries cytokines, chemokines, growth factors, interleukins, and other proteins that may shed insight into the health of a joint. FIG. 1A provides an example analysis of protein concentrations in synovial fluid samples from patients with osteoarthritis.

As used herein, viscosity is a measure of a fluid’s resistance to deformation at a given rate and can be measured in a steady shear test. The storage modulus determines the solid-like character of the fluid. When the storage modulus is high, the more difficult it is to break down the fluid. The reverse is true for a low storage modulus. The loss modulus determines the liquid-like character of the fluid. In general, when storage modulus is high, loss modulus is low, and vice versa.

In some embodiments, the present disclosure relates to a simulated synovial fluid (simSF) product 100 that has viscoelastic properties similar to that of synovial fluid samples from patients with osteoarthritis. The rheological behavior of the simSF product can be measured as a function of time, temperature, strain or stress amplitude and frequency. The results obtained provide information about the sample structural properties such as MW, MWD, concentration, crosslinking density for polymers or particle/domain size, shape, interface properties, etc. for the simSF product. This information is important in product development (formulation) to predict product performance and processing behavior of new or modified materials.

In some embodiments, an exemplary simSF product 100 comprises two or more cytokines, and one or more of a sulfated glycosaminoglycan or a viscosity agent.

Cytokines can include proinflammatory and anti-inflammatory cytokines. Proinflammatory cytokines are endogenous polypeptides that are primarily derived from immune system cells, exert a variety of powerful biological effects and can mediate various immune responses. In an example simSF product, cytokines can include two or more cytokines such as, for example, basic fibroblast growth factor (bFGF), epidermal growth factor (EGF), eotaxin, granulocyte-colony stimulating factor (G-CSF), granulocyte-macrophage colony-stimulating factor (GM-CSF), hepatocyte growth factor (HGF), IFN-alpha, IFN-gamma, IL-1beta, IL-2, IL-2R, IL-4, IL-6, IL-7, IL-8, IL-12, IL-13, IL-15, IL-17A, IL-18, interferon gamma-induced protein 10 (IP-10), monocyte chemoattractant protein-1 (MCP-1), monokine induced by interferon-gamma (MIG), macrophage inflammatory protein-1 alpha (MIP-1alpha), macrophage inflammatory protein-1 beta (MIP-1beta), regulated on activation-normal T cell expressed and secreted (RANTES), tumor necrosis factor alpha (TNF-alpha), or vascular endothelial growth factor (VEGF).

Sulfated glycosaminoglycans or structural carbohydrates, are linear polysaccharides that are made of repeating disaccharide units of N-acetylhexosamine and uronic acid. SimSF product 100 can include a sulfated glycosaminoglycan, including keratan sulfate, chondroitin sulfate, heparin/heparan sulfate, dermatan sulfate, or hyaluronic acid. As sulfated glycosaminoglycans are important structural components of cartilage and provide resistance to compression, they can add functional properties to the simSF product when combined with the two or more cytokines.

Viscosity agent can be a thickening agent that increases the viscosity of a liquid without substantially changing the properties of the product. In some embodiments, simSF product 100 can include hyaluronic acid as a viscosity agent. The molecular weight of the hyaluronic acid can change the viscosity of the simSF product. In some embodiments, simSF product 100 can include a low molecular weight viscosity agent (e.g., equal to or less than 0.3 megadaltons (MDa)) in addition to a high molecular weight viscosity agent (e.g., equal to or greater than 0.3 megadaltons (MDa)). As would be appreciated by one of skill in the art, adjusting the amount of each of low molecular weight viscosity agent and high molecular weight viscosity agent can alter the viscosity of the simSF product as a whole.

FIG. 1B is a heatmap of a first group of patient-derived synovial fluid that demonstrates the large variability among synovial fluid of patients with osteoarthritis. As such, the simSF product 100 may be adjusted depending on the type of osteoarthritis or other characteristics of the patient population of interest. In some embodiments, simSF product may include two or more cytokines that are the highest prevalent cytokines found in a patient-derived synovial fluid. For instance, a first group of subjects with wrist osteoarthritis may present high concentrations of IL-1β and TNF-α that may be the same or different than a second group of subjects with knee osteoarthritis (e.g., IL-1β, TNF-α and IL-6).

In some embodiments, the simSF product 100 may be made to include two or more of the highest prevalent cytokines in the particular group of subjects. As an example, a set of common and prevalent proteins were identified in a first group of 14 patient-derived synovial fluid samples. Concentrations of 30 detected proteins were normalized to the total protein concentrations of the corresponding patient-derived synovial fluid to reveal 5 proteins that were common and most prevalent throughout the first group of patient-derived synovial fluid. The prevalent cytokines can include basic fibroblast growth factor (FGFb, also known as FGF-2), granulocyte-colony stimulating factor (G-CSF), hepatocyte growth factor (HGF), monokine induced by interferon gamma (MIG, also known as CXCL9), and monocyte chemoattractant protein-1 (MCP-1, also known as CXCL2).

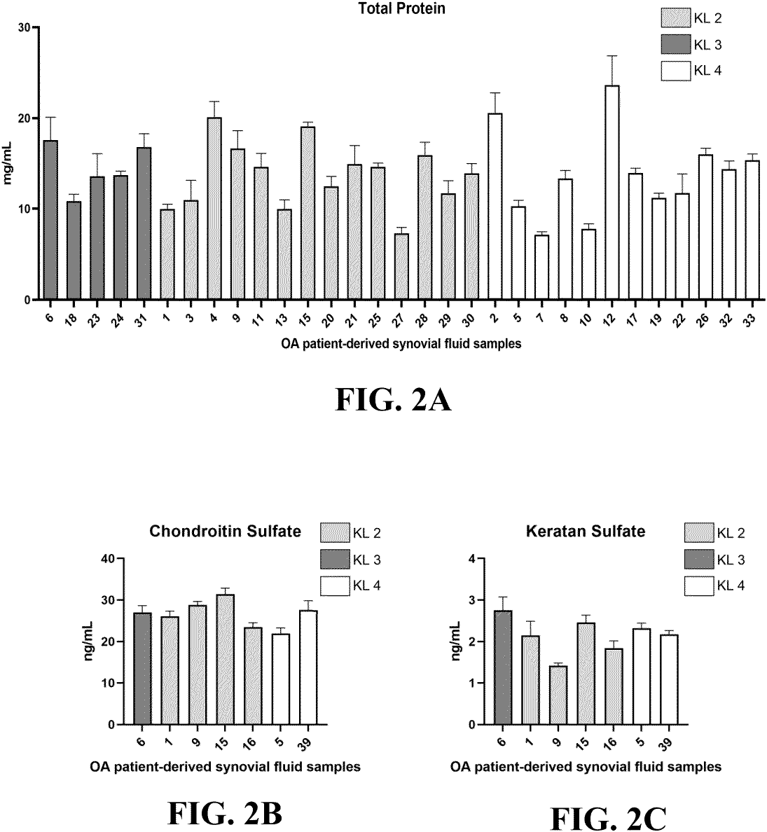

Patient-derived synovial fluid is a protein-rich fluid. FIGS. 2A through 2C illustrate total protein, chondroitin sulfate, and keratan sulfate concentrations for select patients based on the KL score.

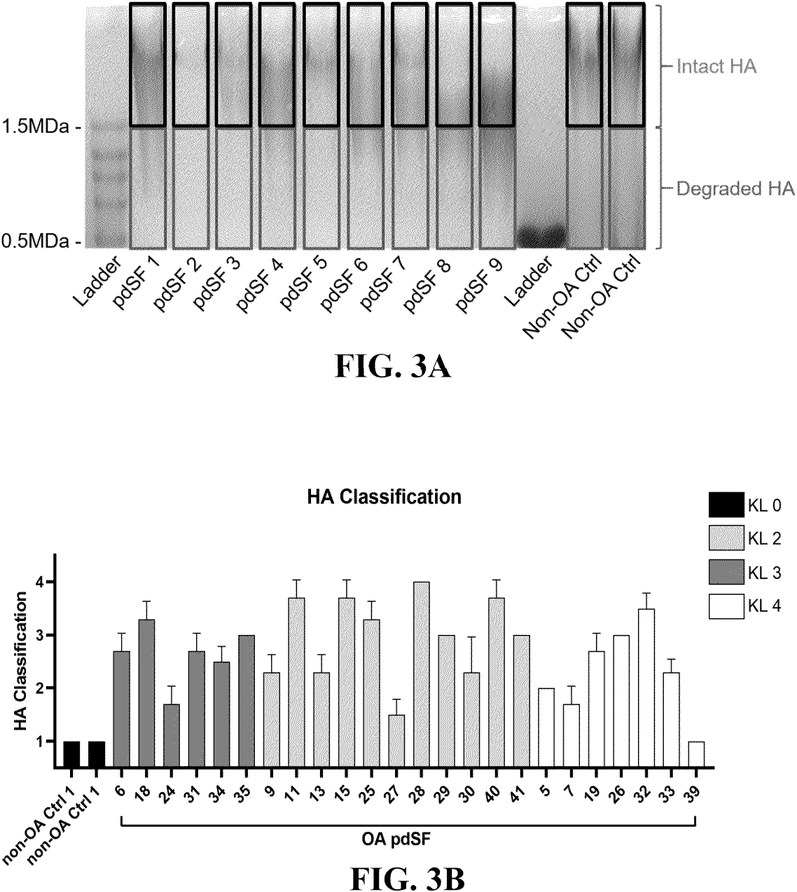

FIGS. 3A and 3B provide classification of hyaluronic acid (HA) degradation in synovial fluid of subjects with osteoarthritis, with FIG. 3B organized by KL score. For HA classification, a grade of 1 means no degradation and all detected HA is intact. A grade of 2 means the presence of intact HA is approximately 4 MDa and the intensity or amount of intact HA is decreased by approximately 20%. A grade of 3 means the presence of degraded HA is equal to a less than approximately 1 MDa and the intensity or amount of intact HA is decreased by approximately 50%. A grade of 4 means the presence of HA is not discernable and the intensity or amount of intact HA is equal to or less than 10% (90% or more is degraded).

SimSF product 100 can also include a blood derivative, such as serum albumin from humans or other animals. The blood derivative can make up a majority of the composition. The blood derivative from human can include human platelet lysate, human serum (whole serum), human plasma. Alternatively, or in addition thereto, blood derivative from animals such as bovine, sheep, goat, chicken, rabbit, rat, or mouse can include serum albumin and serum plasma. For instance, human serum albumin (HSA) can include between about 50% to about 95% based on the total weight per volume of the composition. As shown in Tables 1-3, an example simSF product can include approximately 88% HSA, approximately 10% of viscosity agent, and less than approximately 2% of cytokines, sulfated glycosaminoglycans, and low molecular weight hyaluronic acid.

TABLE 1

Composition of example simSF
Composition of simulated synovial fluid in phosphate buffered saline (PBS)

| | |
|---|---|
| Human serum albumin | 25 mg/mL |
| Basic Fibroblast Growth Factor (bFGF) | 120 ng/mL |
| Granulocyte-Colony Stimulating Factor (G-CSF) | 30 ng/mL |
| Hepatocyte Growth Factor (HGF) | 26 ng/mL |
| Monocyte Chemoattractant Protein-1 (MCP-1) | 19 ng/mL |
| Monokine Induced by Interferon-Gamma (MIG) | 18 ng/mL |
| Chondroitin Sulfate (CS) | 4 µg/mL |
| Keratan Sulfate (KS) | 20 µg/mL |
| Low MW hyaluronic acid | 100 µg/mL |
| High MW hyaluronic acid | 3 mg/mL |

TABLE 2

Composition of example simSF
Composition of simulated synovial fluid in phosphate buffered saline (PBS)

| | |
|---|---|
| Human serum albumin | 30 mg/mL |
| Basic Fibroblast Growth Factor (bFGF) | 120 ng/mL |
| Granulocyte-Colony Stimulating Factor (G-CSF) | 30 ng/mL |
| Hepatocyte Growth Factor (HGF) | 26 ng/mL |
| Monocyte Chemoattractant Protein-1 (MCP-1) | 19 ng/mL |
| Monokine Induced by Interferon-Gamma (MIG) | 18 ng/mL |
| Chondroitin Sulfate (CS) | 4 µg/mL |
| Keratan Sulfate (KS) | 20 µg/mL |
| Low MW hyaluronic acid | 95 µg/mL |
| High MW hyaluronic acid | 5 mg/mL |

TABLE 3

Composition of example simSF
Composition of simulated synovial fluid in phosphate buffered saline (PBS)

| | |
|---|---|
| Human serum albumin | 20 mg/mL |
| Basic Fibroblast Growth Factor (bFGF) | 120 ng/mL |
| Granulocyte-Colony Stimulating Factor (G-CSF) | 30 ng/mL |
| Hepatocyte Growth Factor (HGF) | 26 ng/mL |
| Monocyte Chemoattractant Protein-1 (MCP-1) | 19 ng/mL |
| Monokine Induced by Interferon-Gamma (MIG) | 18 ng/mL |
| Chondroitin Sulfate (CS) | 4 µg/mL |
| Keratan Sulfate (KS) | 20 µg/mL |
| Low MW hyaluronic acid | 105 µg/mL |
| High MW hyaluronic acid | 1 mg/mL |

Of the cytokines, bFGF may make up at least 40% based on the total weight per volume of cytokines in the composition. In some embodiments, bFGF may be 56% based on the total weight per volume of cytokines with the G-CSF, HGF, MCP-1, and MIG making up the remaining 44%, based on the total weight per volume of cytokines in the composition.

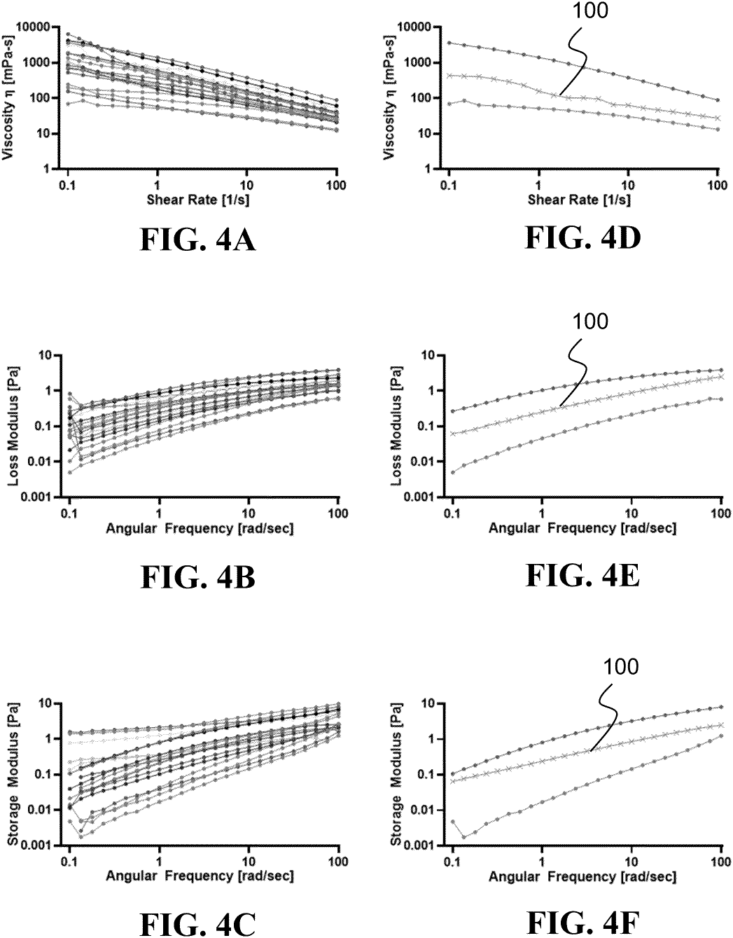

In some embodiments, simSF product 100 can have viscoelastic properties with measurements of viscosity, storage moduli, and loss moduli closely resembling patient-derived synovial fluid. FIGS. 4D through 4F compare viscoelastic properties of simSF product 100 to that of patient derived synovial fluid (min and max samples shown in FIGS. 4D through 4F, and all samples in FIGS. 4A through 4C).

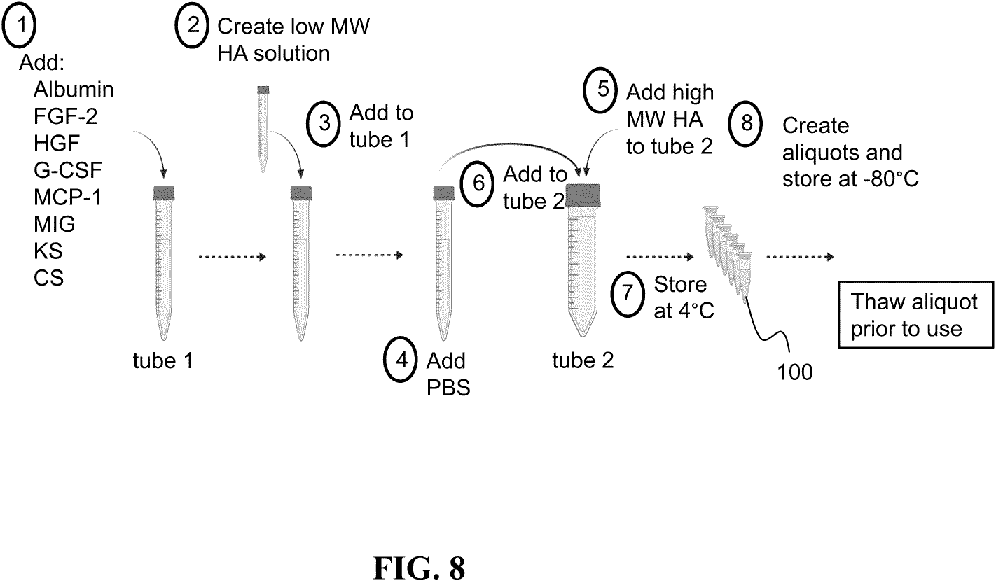

In some embodiments, simSF product 100 can be manufactured as shown in FIG. 8. In general, the composition can be created by mixing HSA with cytokines and sulfated glycosaminoglycans in a first tube. Buffer and/or viscosity agent can be mixed together in a first tube or can be combined in a second tube that is later mixed with the contents in the first tube. The mixture can then be incubated for a predetermined time at a temperature ranging from about 0° C. to about 10° C., preferably 4° C. The predetermined time can range from about 2 hours to about 72 hours, preferably at least about 48 hours. The incubation period may allow for the mixture to crosslink and achieve the appropriate viscoelastic properties of simSF product 100.

In some embodiments, the desired viscoelastic properties is similar to that of a patient-derived synovial fluid. For instance, simSF product 100 can achieve a viscosity ranging from approximately 100 millipascal second (mPa·s) to approximately 10,000 mPa·s when the shear rate is approximately 0.1 reciprocal seconds (s⁻¹). When the shear rate increase from 0.1 s⁻¹ to 1 s⁻¹, to 10 s⁻¹, or to 100 s⁻¹, the viscosity can decrease, as depicted in FIG. 4D. For a shear rate of approximately 1 s⁻¹, the composition comprises a viscosity ranging from approximately 80 millipascal second (mPa·s) to approximately 5,000 mPa·s. For a shear rate of approximately 10 s⁻¹, the composition comprises a viscosity ranging from approximately 70 millipascal second (mPa·s) to approximately 2,000 mPa·s. For a shear rate of approximately 100 s⁻¹, the composition comprises a viscosity ranging from approximately 40 millipascal second (mPa·s) to approximately 1,000 mPa·s.

In the same way, simSF product 100 can achieve a loss modulus similar to that of a patient-derived synovial fluid, as depicted in FIG. 4E. In particular, with an angular frequency of approximately 0.1 radians per second (rad/sec), the composition comprises a loss modulus ranging from approximately 0.01 pascal (Pa) to approximately 1 Pa. At an angular frequency of approximately 1 rad/sec, the composition comprises a loss modulus ranging from approximately 0.1 Pa to approximately 1 Pa. At an angular frequency of approximately 10 rad/sec, the composition comprises a loss modulus ranging from approximately 0.1 Pa to approximately 1.8 Pa. At an angular frequency of approximately 100 rad/sec, the composition comprises a loss modulus ranging from approximately 0.5 Pa to approximately 8 Pa.

As shown in FIG. 4F, the storage modulus of simSF product 100 can reflect that of an average patient-derived synovial fluid. With an angular frequency of approximately 0.1 rad/sec, the composition comprises a storage modulus ranging from approximately 0.001 Pa to approximately 1.5 Pa. At an angular frequency of approximately 1 rad/sec, the composition comprises a storage modulus ranging from approximately 0.01 Pa to approximately 1.8 Pa. At an angular frequency of approximately 10 rad/sec, the composition comprises a storage modulus ranging from approximately 0.1 Pa to approximately 5 Pa. At an angular frequency of approximately 100 rad/sec, the composition comprises a storage modulus ranging from approximately 0.8 Pa to approximately 10 Pa.

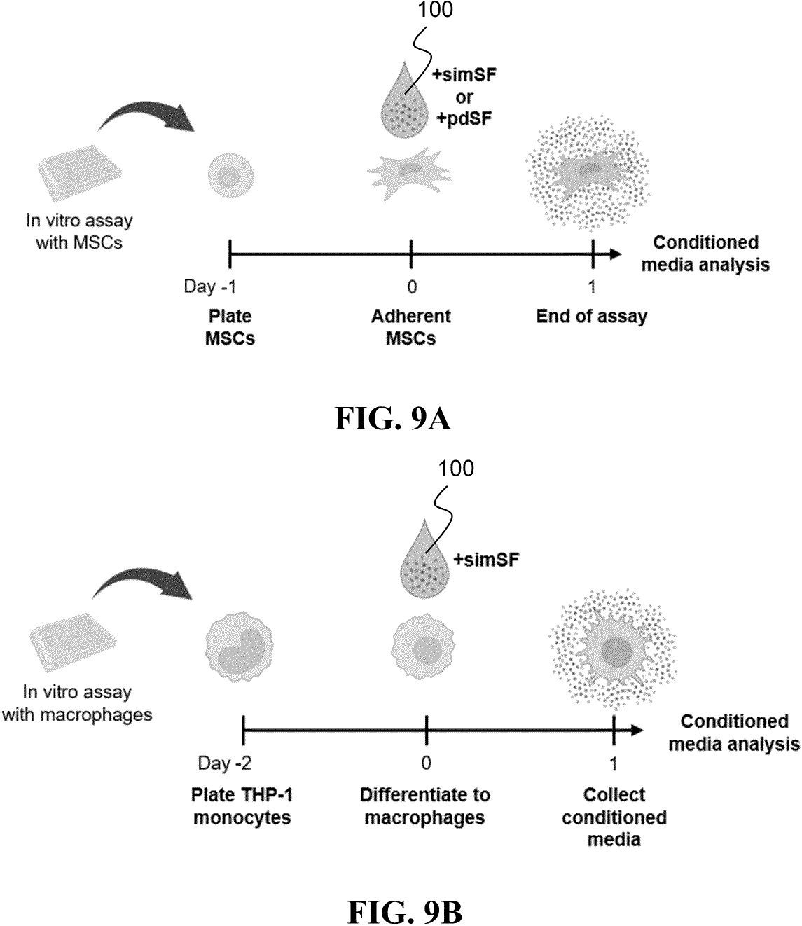

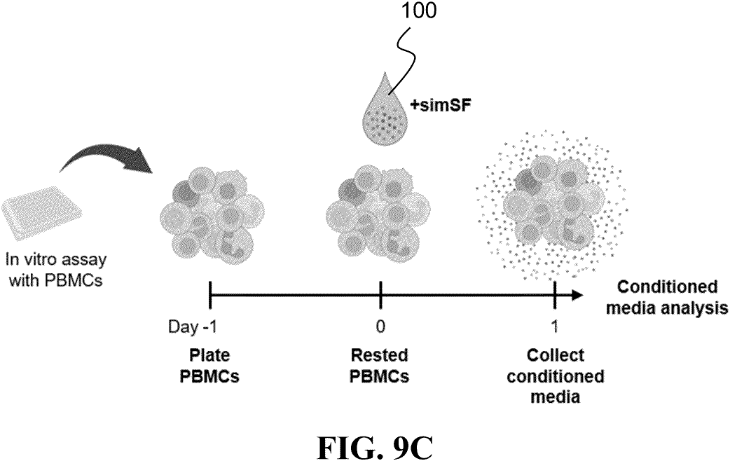

In some embodiments, 2D and 3D microfluidic synovial fluid exposure potency assays can determine the comparability of the final simSF product 100 to patient-derived synovial fluid. FIGS. 9A through 9C provide schematic illustrations of in vitro assays with simSF product 100. FIG. 9A is an in vitro osteoarthritis-targeted potency assay used to evaluate bone marrow-derived mesenchymal stromal cell (MSC) therapy responses to either patient-derived synovial fluid or simSF product 100. As shown, either the patient-derived synovial fluid or the simSF product 100 can be contacted with adherent MSCs. At the end of the assay (e.g., after day 1 of incubating the MSCs with either patient-derived synovial fluid or the simSF product 100, the conditioned media is analyzed for cell therapy responses.

Similarly, FIG. 9B illustrates in vitro osteoarthritis-targeted potency assay used to evaluate differentiation to macrophages. SimSF product 100 can be contacted with macrophages, or THP-1 monocytes and analyzed at the end of the assay for cell differentiation.

FIG. 9C illustrates in vitro assay with PBMCs to test comparability of simSF product 100 as a pharmacological screening tool. SimSF product 100 can be contacted with rested PBMCs and analyzed at the end of the assay for the ability of simSF to mimic cellular responses of PBMCs to patient-derived synovial fluid in vitro.

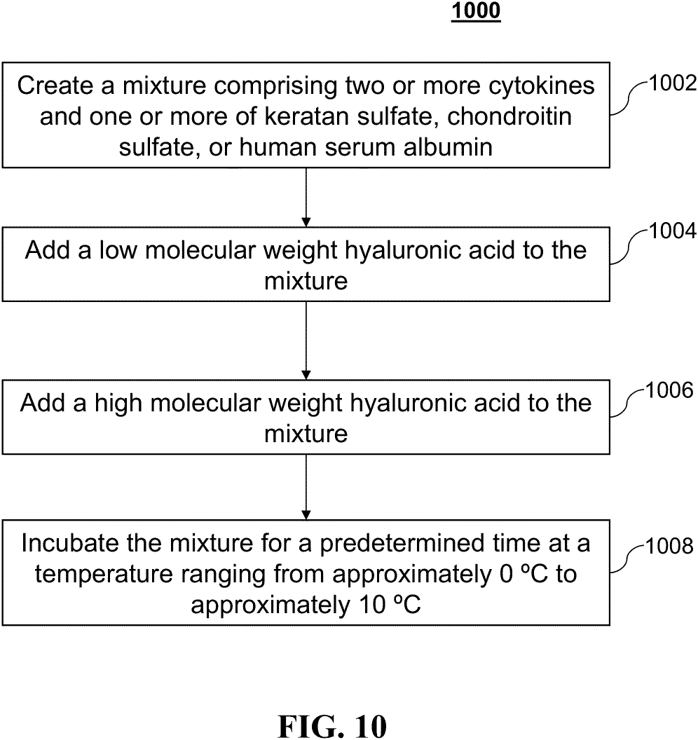

FIG. 10 is a flowchart of a method 1000 of manufacturing an example simulated synovial fluid. The method 1000 can include creating a mixture comprising two or more cytokines and one or more of keratan sulfate, chondroitin sulfate, or human serum albumin at step 1002. The method 1000 can further include adding a low molecular weight hyaluronic acid to the mixture at step 1004. In addition, the method 1000 includes adding a high molecular weight hyaluronic acid to the mixture at step 1006. After mixing all components together, method 1000 can include incubating the mixture for a predetermined time at a temperature ranging from approximately 0° C. to approximately 10° C. at step 1008. The predetermined time can range from approximately 2 hours to approximately 72 hours. Method 1000 can stop at step 1008 or can optionally include storing the mixture for later use by thawing the mixture prior to use. In addition, method 1000 can optionally include obtaining one or more samples of patient-derived synovial fluid from one or more patients and analyzing a protein content of the one or more samples of patient-derived synovial fluid.

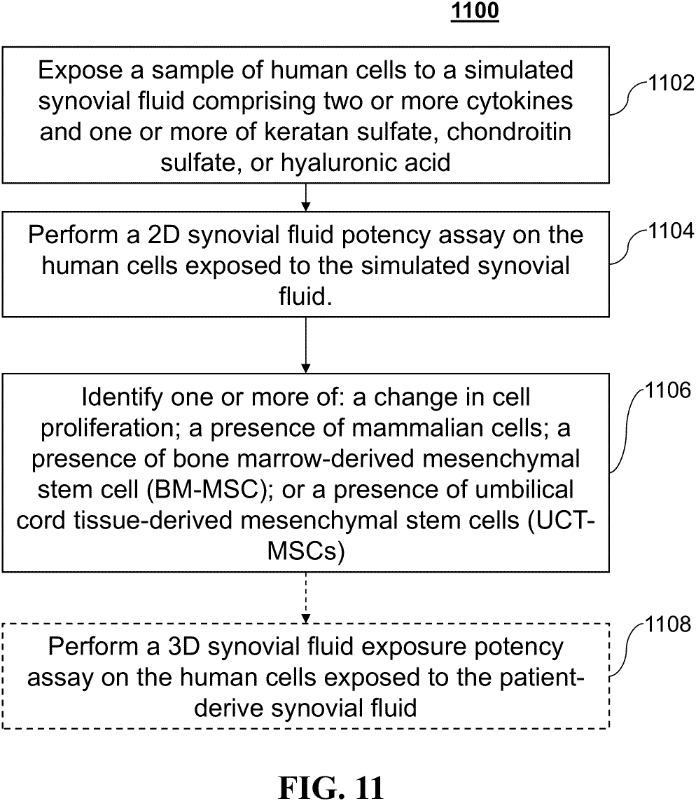

FIG. 11 is a flowchart of a method 1100 of analyzing one or more cellular responses from mammalian cells using an example simulated synovial fluid. Method 1100 can include exposing the sample of human cells to a simulated synovial fluid at step 1102. The simulated synovial fluid can comprise two or more cytokines and one or more of keratan sulfate, chondroitin sulfate, or hyaluronic acid. Method 1100 can further include performing a 2D synovial fluid potency assay on the human cells exposed to the simulated synovial fluid at step 1104. Method 1100 can also include identifying one or more of: a change in cell proliferation; a presence of mammalian cells; a presence of bone marrow-derived mesenchymal stem cell (BM-MSC); or a presence of umbilical cord tissue-derived mesenchymal stem cells (UCT-MSCs) at step 1106. Method 1100 can stop at step 1106 or can optionally include performing a 3D microfluidic synovial fluid exposure potency assay on the human cells exposed to the patient-derive synovial fluid at step 1108.

The following examples further illustrate aspects of the present disclosure. However, they are in no way a limitation of the teachings or disclosure of the present disclosure as set forth herein.

### EXAMPLES

Osteoarthritis patient-derived synovial fluid was derived using a human 30-Plex cytokine panel Luminex™ kit to elucidate the cytokine makeup across varying severities of osteoarthritis disease. From the cytokine panel, a simulated synovial fluid (simSF) product was developed. Like plasma, synovial fluid contains highly abundant proteins such as albumin. Although synovial fluid is rich in hyaluronic acid and lubricin which provides lubrication to synovial joints, findings have suggested that low abundance proteins may be important in both osteoarthritis pathogenesis and biomarker discovery. Challenges regarding variability of synovial fluid composition among donors arise from differences in osteoarthritis pathogenesis and progression which can be influenced by risk factors, e.g. sex, body mass index, and lifestyle of individuals. Using a targeted panel of numerous proteins that function as cytokines, chemokines and growth factors, characterization of osteoarthritis patient-derived synovial fluid may reveal key signaling molecules that are common and abundant in patients with knee osteoarthritis. For the development of a simSF product, key signaling molecules can recreate similar functions and outcomes in target cells as those that occur with patient-derived synovial fluid. The results of patient-derived synovial fluid characterization (n=30), identification of key proteins, development of initial simSF prototypes, and cell secretory outcomes were evaluated when exposed to patient-derived synovial fluid and simSF. Additional characterization was implemented, including quantitative analysis of hyaluronic acid, chondroitin sulfate, keratan sulfate, and rheological measurements. Ultimately, the comprehensive analysis of patient-derived synovial fluid informed the integral properties of a developed simSF product that mimics synovial fluid from individuals with knee osteoarthritis.

Osteoarthritis patient samples of synovial fluid and cell therapies, i.e. bone marrow aspirate concentrate (BMAC), umbilical cord tissue-derived Mesenchymal Stromal Cells (UCT-MSCs), were provided by the MILES clinical trial (NCT03818737). Additionally, BMAC-derived Mesenchymal Stromal Cells (BM-MSCs) were isolated and culture-expanded for analysis. To evaluate cell therapies with greater reproducibility of predictive outcomes, targeted 2D and 3D microfluidic potency assays containing a developed simulated osteoarthritis synovial fluid (simSF) were designed that was informed by osteoarthritis patient-derived synovial fluid (pdSF) analyses. Initially, patient-derived synovial fluid was comprehensively characterized for proteomic and viscoelastic properties to inform the development of simSF prototypes. Then, a final simSF product was added to 2D and 3D microfluidic potency assays to mount responses in cell therapies. Following the potency assays, cell proliferation was measured, and conditioned media was analyzed using a targeted secretome assay to evaluate the predictive outcomes of each cell therapy. As a result, responses were cell type-specific following simSF exposure in both potency assays. The profile of secreted proteins from each cell type was also assay-specific (i.e., 2D versus 3D microfluidic). Results generated outcome criteria that were used to evaluate cell therapies in response to osteoarthritis synovial fluid.

### Example 1: Hyaluronidase Treatment of Patient-Derived Synovial Fluid

Vials of patient-derived synovial fluid (n=30) and a reference synovial fluid were thawed at room temperature and then centrifuged at 1000xg for 10 minutes. For each patient-derived synovial fluid, 500 µL of supernatant was transferred to microcentrifuge tubes and incubated with 4 mg/mL hyaluronidase solution (v/v) for 15 min at 37° C. Samples were vortexed for 5 seconds and then incubated for an additional 15 min at 37° C. Samples were centrifuged for 1000xg for 5 minutes and supernatants were collected and directly used in Luminex assays.

### Example 2: Synovial Fluid Characterization Using Luminex™ Assay

Hyaluronidase treated patient-derived synovial fluid samples and reference synovial fluid were analyzed using Invitrogen™ Cytokine 30-Plex Human Panel Luminex™ (LHC6003M, Thermo Fisher Scientific) according to manufacturer’s instructions. Briefly, lyophilized standards were reconstituted and serially diluted to prepare 8 working standards. Luminex™ plates containing magnetic antibody beads were washed and then standards (2 technical replicates prepared) or samples of patient-derived synovial fluid (4 technical replicates prepared) or reference synovial fluid (4 technical replicates prepared) were added to wells for overnight incubation at 4° C. under mild agitation. Using a magnetic plate, Luminex™ plates samples were decanted, the beads washed twice, and then incubated with biotinylated detector antibody for 1 hour at room temperature under mild agitation and protected from light. Using a magnetic plate, Luminex™ plates were decanted, the beads washed twice, and then incubated with streptavidin-RPE solution for 30 minutes at room temperature under mild agitation and protected from light. Next, Luminex™ plates were washed three times using the magnetic plate, reconstituted and were read using Luminex™ xMAP™ System on a Bioplex-200 plate reader (BioRad).

### Example 3: Total Protein Assay

Total protein of patient-derived synovial fluid samples was measured using Pierce™ BCA Protein Assay Kit according to manufacturer’s instruction using the microplate procedure. Albumin standards were prepared by serial dilutions and added to wells of a 96-well plate (2 technical replicates prepared). patient-derived synovial fluid samples were diluted 1:10 in PBS and then added to the wells of a 96-well plate (3 technical replicates prepared). Wells containing standards and patient-derived synovial fluid were incubated with working reagent for 30 minutes at 37° C. Following incubation, plates were allowed to cool for 5 minutes and then read using a standard plate reader under 562 nm absorbance.

### Example 4: 2D Synovial Fluid Exposure Potency Assay

UCT-MSC, BMAC, or BMAC-MSCs were thawed, counted, and then prepared at a concentration of 8.0×10⁵ cell/mL in XSFM media (Irvine Scientific/Fujifilm). Using a 96-well plate, 100 µL of cell suspension was plated into each well (4 technical replicates prepared) and then placed in a humidified incubator set to 37° C. and 5% CO₂. After 24 hours, 20% patient-derived synovial fluid or 20% simSF was prepared in XSFM media, and 100 µL of either patient-derived synovial fluid or simSF was added to the cells in each well designated for patient-derived synovial fluid or simSF exposure, resulting in exposure of the cells to a final formulation of 10% patient-derived synovial fluid or 10% simSF. 100 µL of XSFM alone was also added to cells designated as basal media controls. The plate was placed in a humidified incubator for 24 hours, and then conditioned media was collected and stored at -20° C. for Luminex analysis.

### Example 5: 3D Microfluidic Synovial Fluid Exposure Potency Assay

BM-MSC cells were encapsulated in PEG-4MAL hydrogels with 1 mM functionalized RGD peptide. 8.0×10⁴ cells designated per 3D samples. The cell-laden hydrogels were placed in the PDMS devices and perfused with XSFM base media prepared with 10% patient-derived synovial fluid, 10% simSF, without synovial fluid (basal media control), tumor necrosis factor-α, or interleukin-1β. Media perfusion was 1.0 µL per minute for 24 hours. Conditioned media was collected for Luminex analysis.

### Example 6: Analysis and Results

Raw data from Luminex™ Assays was imported into JMP 15 software for analysis and to generate Zscores, unsupervised hierarchical clustering, and principal component analysis plots. Heat maps of Zscores were created using GraphPad Prizm software.

### Example 7: Protein Composition of Patient-Derived Synovial Fluid Shows Variabilities Among Donors

The 30-Plex Cytokine Luminex™ was used to quantify cytokines, chemokines and growth factors present in 14 patient-derived synovial fluid for preliminary characterization from 2 subsequent assays. A sample of the same reference synovial fluid was run in each assay for batch comparisons. Individual targeted proteome values were standardized in JMP Statistical software for Z-scores and plotted in Prism (FIG. 1B) as average Zscore for the range of proteins listed. Of note, total protein concentration of the patient-derived synovial fluid did not directly correlate to high proteome concentrations (FIG. 1B). Considering KL scores for each donor, the data suggest large variability of protein composition and total protein among donors that is also not correlative to disease severity. In general, for this preliminary analysis of patient-derived synovial fluid, the KL grades 2 and 4 appeared to reflect lower protein concentrations than the KL 3 samples. Both pro-inflammatory and anti-inflammatory proteins were measured in the protein composition of patient-derived synovial fluid.

Analysis of the normalized protein concentrations according to KL score supported the top 5 proteins as common and most prevalent proteins in patient-derived synovial fluid regardless of disease severity. In most cases, the concentrations of the top 5 proteins showed increasing trend in concentrations with increasing KL score. Together, this data supported the decision the include FGF-2, G-CSF, HGF, MIG, and MCP-1 in the early simSF prototypes.

### Example 8: Analysis of 30 Patient-Derived Synovial Fluid Shows Commonalities and Variabilities in Protein Components

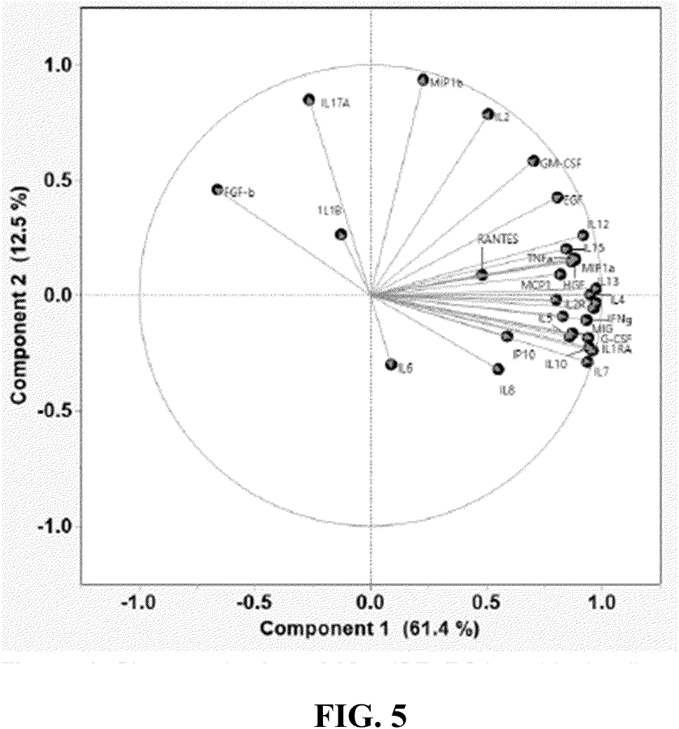

All 30 patient-derived synovial fluid were then analyzed using multivariate principal component analysis (PCA) in JMP (FIG. 5). The PCA biplot reduced the number of dimensions to a few principal components which describe the main variation in the patient-derived synovial fluid samples. The loading matrix provides a measure of strength and direction for each protein and its ability to influence the principal component. Some key proteins such as IL-13, IL-7, G-CSF, listed below in Table 4, strongly influenced principal component 1 (presenting above 0.8700, while MIP-1 beta was more influential in principal component 2 (presenting at 0.93633). Two variables that appear close to one another, such as IL-12 and IL-15, are positively correlated but those at 90° are not correlated. The large angle between FGFb and IL-4 suggested that they were negatively correlated. Principle component 1 explained 61.4% of the variation in the targeted proteome of the patient-derived synovial fluid samples.

TABLE 4

| Protein | Principle component 1 | Principle component 2 |
|---|---|---|
| EGF | 0.80692 | 0.42607 |
| Eotaxin | 0.97404 | -0.03171 |
| FGF-b | -0.66202 | 0.45972 |
| G-CSF | 0.94007 | -0.18420 |
| GM-CSF | 0.70426 | 0.58387 |
| HGF | 0.88455 | 0.15594 |
| IFNa | 0.96396 | -0.5532 |
| IFNq | 0.93295 | -0.10675 |
| IL10 | 0.94404 | -0.22586 |
| IL12 | 0.91791 | 0.26000 |
| IL13 | 0.97359 | 0.02916 |
| IL15 | 0.84546 | 0.20121 |
| IL17A | -0.26596 | 0.84979 |
| IL1RA | 0.95779 | -0.23977 |
| 1L1B | -0.12744 | 0.26383 |
| IL2 | 0.50847 | 0.78687 |
| IL2R | 0.83013 | -0.09075 |
| IL4 | 0.94817 | 0.00340 |
| IL5 | 0.85873 | -0.17984 |
| IL6 | 0.08842 | -0.29989 |
| IL7 | 0.93728 | -0.28854 |
| IL8 | 0.55195 | -0.32021 |
| IP10 | 0.59010 | -0.17751 |
| MCP1 | 0.81956 | 0.09106 |
| MIG | 0.87115 | -0.16512 |
| MIP1a | 0.86898 | 0.14614 |
| MIP1b | 0.22676 | 0.93633 |
| RANTES | 0.48119 | 0.08815 |
| TNFa | 0.87856 | 0.16055 |
| VEGF | 0.80169 | -0.01867 |

Analysis of the concentrations of each protein measure by the 30-Plex Luminex™ assay supported the preliminary analysis. All patient-derived synovial fluid contained the 5 proteins with the highest concentrations detected as FGFb, HGF, G-CSF, MIG, and MCP-1. Each patient-derived synovial fluid sample was color-coded according to the KL score of the knee osteoarthritis severity. No trend was observed that correlated the presence of a protein directly with a particular KL score, although a limited number of subjects and unequal distribution of each of the KL scores exists.

### Example 9: Development of SimSF Prototypes

Protein characterization of the 30 patient-derived synovial fluid samples informed the development of the early prototypes of simulated SF. The analysis provided the top 5 proteins that are present in all 30 patient-derived synovial fluid samples and at highest concentrations, i.e. FGFb, HGF, G-CSF, MIG, and MCP-1. Recombinant proteins of each of the 5 top proteins were purchased to make the early simSF prototypes in concentration ranges that were extrapolated from those measure in the patient-derived synovial fluid (Table 5).

TABLE 5

Simulated synovial fluid (in PBS)

| Proteins | Grade 1 [low] | Grade 2 | Grade 3 | Grade 4 [high] |
|---|---|---|---|---|
| HSA (mg) | 25 | 25 | 25 | 25 |
| FGFb (ng) | 12 | 40 | 80 | 120 |
| G-CSF (ng) | 3 | 10 | 20 | 30 |
| HGF (ng) | 2.6 | 8.7 | 17.3 | 26 |
| MCP-1 (ng) | 1.9 | 6.3 | 12.7 | 19 |
| MIG (ng) | 1.8 | 6 | 12 | 18 |

To represent the high abundance proteins from the blood that concentrate SF, human serum albumin (HSA) was added along with the top 5 proteins as an example simSF prototype in phosphate buffered saline (PBS) as the vehicle. These prototypes were developed with the proteins with high concentrations that are classified as cytokines, chemokines, and growth factors with known signaling functions. Initial pilots using the developed 2D and 3D microfluidic synovial fluid exposure potency assays were performed in order to compare secretory responses from target cells when exposed to the simSF prototypes and patient-derived synovial fluid.

Patient-derived synovial fluid has viscoelastic properties that were not initially considered as a necessary factor to mimic in the simSF product. However, after analyzing the size distribution of hyaluronic acid (HA), a better simSF product was created. HA is one of the most abundant proteins in synovial fluid with high molecular weight (MW) HA as most abundant in synovial fluid from healthy and OA patients. High MW HA provides the viscosity of synovial fluid; thus rheological measurements of the patient-derived synovial fluid were implemented to inform the concentration of high MW HA that to be used in the simSF products. Low MW HA is known to be pro-inflammatory, and more prevalent in osteoarthritis synovial fluid compared to healthy synovial fluid. Chondroitin sulfate (CS) and keratan sulfate (KS) are putative biomarkers of osteoarthritis in synovial fluid. Ongoing analysis of viscoelastic properties, HA, CS, and KS were performed on patient-derived synovial fluid that extended the characterization of patient-derived synovial fluid and better informed the simSF products.

### Example 10: Testing of Simulated Synovial Fluid Prototypes Using 2d and 3d Microfluidic Potency Assays Demonstrate Comparability to Patient-Derived Synovial Fluid

A number of potency assay pilots were used to optimize conditions and operating procedures for the 2D synovial fluid exposure potency assays. Target cells used in the 2D and 3D microfluidic synovial fluid exposure potency assays included umbilical cord tissue-derived mesenchymal stromal cells (UCT-MSCs), bone marrow aspirate concentrate (BMAC) cells, and BMAC-derived mesenchymal stromal cells (BMAC-MSCs). The results showed that a 24 hour exposure of thawed MSCs to patient-derived synovial fluid (10%, 25% and 50% in culture medium) compromised viability and higher concentrations of patient-derived synovial fluid led to less adherence and lower viability. This suggested that a 24 hour culture rescue period was required for MSCs adherence prior to exposure and incubation with patient-derived synovial fluid.

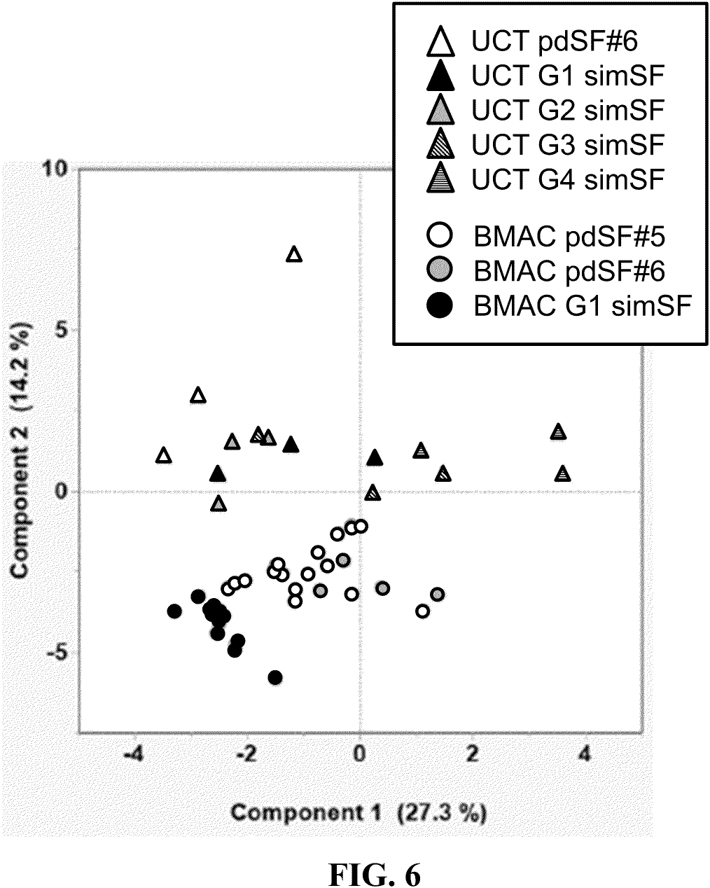

The next pilot implemented the 24 hour culture rescue period. By use of linear discriminant analysis, the results determined that the targeted MSC secretome from 10% patient-derived synovial fluid incubation was similar to that from 25% or 50% patient-derived synovial fluid, thus incubation of the target cells with 10% patient-derived synovial fluid or simSF was implemented. Furthermore, using PCA, the responses for BMAC, BMAC-MSC or UCT-MSC were similar for 1 day or 3 days incubation for their respective cell types, in 10% patient-derived synovial fluid. Therefore the protocol for the 2D and 3D microfluidic synovial fluid exposure potency assays were modified to incubation with synovial fluid for 1 day and subsequent collection of the conditioned media that would be stored at -20° C. for Luminex™ analysis. Differences in secretome for BMAC-MSC, UCT-MSC, and BMAC were measured, suggesting that the next steps in developing a panel of proteins that will be analyzed for criteria regarding the quality of cells will be distinguishable for each cell therapy type. More importantly, development of example simSF prototypes and testing in parallel to patient-derived synovial fluid in the 2D SF exposure potency assay demonstrated similarity of secretory responses from both UCT-MSC and BMAC (FIG. 6).

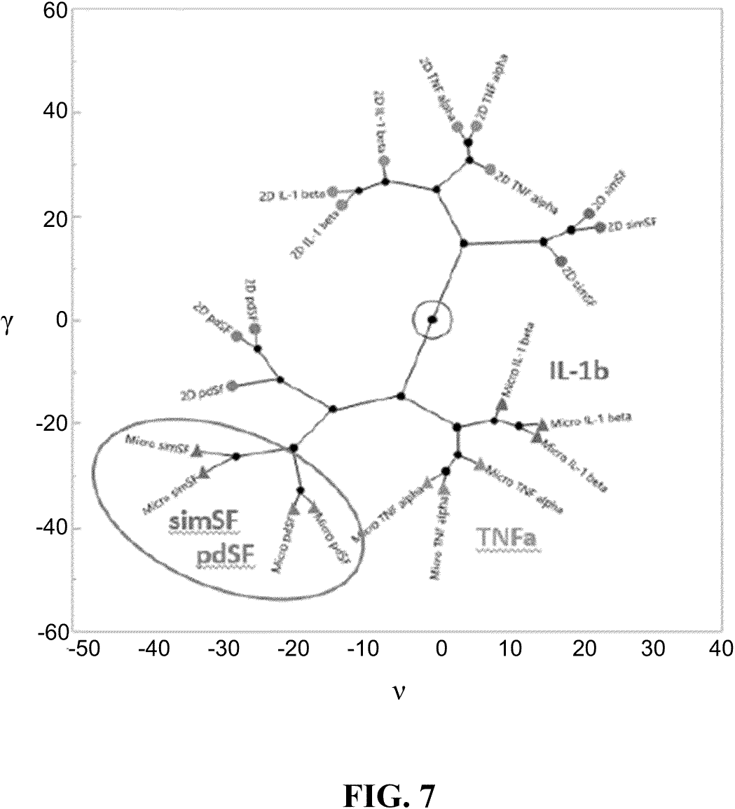

Pilots were also used to establish the conditions and procedures for the 3D microfluidic synovial fluid exposure potency assay. Briefly, UCT-MSCs, BMAC, and BMAC-MSCs were embedded in a 3D conformation in a microfluidic device that was assembled using a PDMS casted device. Medias were perfused via a pressure-driven pump. After 24 hrs, target cells secretory responses were found most comparable between simSF and patient-derived synovial fluid, as determined by Hierarchal Clustering Ward Method (JMP15). 3D microfluidic exposure to single molecules, tumor necrosis factor-alpha (TNFa) or interleukin-1 beta (IL-1b) were also included in this analysis for their common use in the field as OA-mimicking stimuli. These results show the Grade 4 simSF prototype mounted similar responses to those mounted by patient-derived synovial fluid in a 3D microfluidic environment (FIG. 7).

Characterization of patient-derived synovial fluid has informed the development of a simSF product. Using an assay that detects 30 proteins, analysis of the protein makeup in patient-derived synovial fluid consistently showed FGFb, G-CSF, HGF, MIG, MCP-1 at the highest concentrations in all patient-derived synovial fluid. Given the known content of high abundance proteins in synovial fluid, the first simSF prototypes contained HSA along with varying concentrations of FGFb, G-CSF, HGF, MIG, MCP-1. Experiments using the 2D and 3D microfluidic potency assays were performed to compare the simSF prototypes to patient-derived synovial fluid. Results from the 2D potency assay revealed secretory responses from target cells, i.e. UCT-MSCs, BMAC, and BMAC-MSCs, that were similar between the simSF and patient-derived synovial fluid. The 3D microfluidic potency assay showed greatest comparability between the secretory responses of bone marrow-derived MSC exposed to patient-derived synovial fluid and simSF and not IL-1b or TNFa. Testing using target cells and measured secretory responses demonstrate the comparability of the simSF product to patient-derived synovial fluid.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

While the present disclosure has been described in connection with a plurality of exemplary aspects, as illustrated in the various figures and discussed above, it is understood that other similar aspects can be used, or modifications and additions can be made to the described aspects for performing the same function of the present disclosure without deviating therefrom. For example, in various aspects of the disclosure, methods and compositions were described according to aspects of the presently disclosed subject matter. But other equivalent methods or compositions to these described aspects are also contemplated by the teachings herein. Therefore, the present disclosure should not be limited to any single aspect, but rather construed in breadth and scope in accordance with the appended claims.

The disclosed technology described herein can be further understood according to the following clauses:

Clause 1: A composition comprising: two or more cytokines, and one or more of keratan sulfate, chondroitin sulfate, or hyaluronic acid; wherein the composition simulates a fluid from a patient.

Clause 2: The composition of Clause 1, wherein at a shear rate of approximately 0.1 reciprocal seconds (s⁻¹), the composition comprises a viscosity ranging from approximately 100 millipascal second (mPa·s) to approximately 10,000 mPa·s.

Clause 3: The composition of Clause 1, wherein at a shear rate of approximately 1 reciprocal seconds (s⁻¹), the composition comprises a viscosity ranging from approximately 80 millipascal second (mPa·s) to approximately 5,000 mPa·s.

Clause 4: The composition of Clause 1, wherein at a shear rate of approximately 10 reciprocal seconds (s⁻¹), the composition comprises a viscosity ranging from approximately 70 millipascal second (mPa·s) to approximately 2,000 mPa·s.

Clause 5: The composition of Clause 1, wherein at a shear rate of approximately 100 reciprocal seconds (s⁻¹), the composition comprises a viscosity ranging from approximately 40 millipascal second (mPa·s) to approximately 1,000 mPa·s.

Clause 6: The composition of Clause 1, wherein at an angular frequency of approximately 0.1 radians per second (rad/sec), the composition comprises a loss modulus ranging from approximately 0.01 pascal (Pa) to approximately 1 Pa.

Clause 7: The composition of Clause 1, wherein at an angular frequency of approximately 1 radian per second (rad/sec), the composition comprises a loss modulus ranging from approximately 0.1 pascal (Pa) to approximately 1 Pa.

Clause 8: The composition of Clause 1, wherein at an angular frequency of approximately 10 radians per second (rad/sec), the composition comprises a loss modulus ranging from approximately 0.1 pascal (Pa) to approximately 1.8 Pa.

Clause 9: The composition of Clause 1, wherein at an angular frequency of approximately 100 radians per second (rad/sec), the composition comprises a loss modulus ranging from approximately 0.5 pascal (Pa) to approximately 8 Pa.

Clause 10: The composition of Clause 1, wherein at an angular frequency of approximately 0.1 radians per second (rad/sec), the composition comprises a storage modulus ranging from approximately 0.001 pascal (Pa) to approximately 1.5 Pa.

Clause 11: The composition of Clause 1, wherein at an angular frequency of approximately 1 radian per second (rad/sec), the composition comprises a storage modulus ranging from approximately 0.01 pascal (Pa) to approximately 1.8 Pa.

Clause 12: The composition of Clause 1, wherein at an angular frequency of approximately 10 radians per second (rad/sec), the composition comprises a storage modulus ranging from approximately 0.1 pascal (Pa) to approximately 5 Pa.

Clause 13: The composition of Clause 1, wherein at an angular frequency of approximately 100 radians per second (rad/sec), the composition comprises a storage modulus ranging from approximately 0.8 pascal (Pa) to approximately 10 Pa.

Clause 14: The composition of Clause 1, the cytokines comprising one or more of basic fibroblast growth factor (bFGF), epidermal growth factor (EGF), eotaxin, granulocyte-colony stimulating factor (G-CSF), granulocyte-macrophage colony-stimulating factor (GM-CSF), hepatocyte growth factor (HGF), IFN-alpha, IFN-gamma, IL-2, IL-2R, IL-6, IL-7, IL-8, IL-12, IL-13, IL-15, IL-17A, interferon gamma-induced protein 10 (IP-10), monocyte chemoattractant protein-1 (MCP-1), monokine induced by interferon-gamma (MIG), macrophage inflammatory protein-1 alpha (MIP-1alpha), macrophage inflammatory protein-1 beta (MIP-1beta), regulated on activation-normal T cell expressed and secreted (RANTES), tumor necrosis factor alpha (TNF-alpha), or vascular endothelial growth factor (VEGF).

Clause 15: The composition of Clause 14, the cytokines comprising bFGF, G-CSF, HGF, MIG, or MCP-1.

Clause 16: The composition of Clause 15, wherein the bFGF comprises at least 40% based on a total weight per volume of cytokines in the composition.

Clause 17: The composition of Clause 15, wherein the bFGF comprises from approximately 50% to approximately 60% based on a total weight per volume of cytokines in the composition.

Clause 18: The composition of any of Clauses 1-17, further comprising human serum albumin (HSA).

Clause 19: The composition of Clause 18, wherein the HSA comprises from approximately 80% to approximately 90%, based on a total weight per volume of composition.

Clause 20: The composition of any of Clauses 1-19, comprising at least one of a high molecular weight hyaluronic acid or a low molecular weight hyaluronic acid.

Clause 21: The composition of any of Clauses 1-19, comprising both a high molecular weight hyaluronic acid and a low molecular weight hyaluronic acid.

Clause 22: The composition of Clause 21, wherein the composition comprises: high molecular weight hyaluronic acid present from approximately 5% to approximately 20%, based on a total weight per volume of the composition; and low molecular weight hyaluronic acid present from approximately 0.01% to approximately 5%, based on a total weight per volume of the composition.

Clause 23: The composition of Clause 21, wherein the high molecular weight hyaluronic acid is present in a higher concentration within the composition compared to the low molecular weight hyaluronic acid.

Clause 24: The composition of any of Clauses 20-23, wherein the high molecular weight hyaluronic acid comprises a molecular weight greater than 0.3 megadaltons (MDa).

Clause 25: The composition of any of Clauses 20-24, wherein the low molecular weight hyaluronic acid comprises a molecular weight equal to or less than 0.3 megadaltons (MDa).

Clause 26: The composition of any of Clauses 1-25, wherein the simulated fluid has a viscosity similar to a viscosity of patient-derived synovial fluid.

Clause 27: The composition of any of Clauses 1-26, wherein the simulated fluid has a storage moduli similar to a storage moduli of patient-derived synovial fluid.

Clause 28: The composition of any of Clauses 1-27, wherein the simulated fluid has a loss moduli similar to a loss moduli of patient-derived synovial fluid.

Clause 29: The composition of any of Clauses 1-28, wherein the simulated fluid is knee osteoarthritis simulated synovial fluid.

Clause 30: The composition of Clause 1, wherein the patient comprises a degenerative joint disease.

Clause 31: The composition of Clause 30, wherein the patient comprises an osteoarthritic condition.

Clause 32: A method of making a composition for simulating a fluid from a patient, the method comprising: creating a mixture comprising two or more cytokines and one or more of keratan sulfate, chondroitin sulfate, or human serum albumin; adding a low molecular weight hyaluronic acid to the mixture; adding a high molecular weight hyaluronic acid to the mixture; and incubating the mixture for a predetermined time at a temperature ranging from approximately 0° C. to approximately 10° C.

Clause 33: The method of Clause 32, further comprising: obtaining one or more samples of patient-derived synovial fluid from one or more patients; and analyzing a protein content of the one or more samples of patient-derived synovial fluid.

Clause 34: The method of Clause 32, further comprising: achieving a viscosity ranging from approximately 100 millipascal second (mPa·s) to approximately 1000 mPa·s, based on a shear rate ranging from approximately 0.1 reciprocal seconds (s⁻¹) to 100 s⁻¹.

Clause 35: The method of Clause 32, further comprising: achieving a loss modulus ranging from approximately 0.01 pascal (Pa) to approximately 3 Pa, based on a frequency ranging from approximately 0.1 radians per second (rad/sec) to approximately 100 rad/sec.

Clause 36: The method of Clause 32, further comprising: achieving a storage modulus ranging from approximately 0.01 pascal (Pa) to approximately 1 Pa, based on a frequency ranging from approximately 0.1 radians per second (rad/sec) to approximately 100 rad/sec.

Clause 37: The method of Clause 32, further comprising achieving a viscosity similar to a viscosity of patient-derived synovial fluid.

Clause 38: The method of Clause 32, further comprising achieving a storage moduli similar to a storage moduli of patient-derived synovial fluid.

Clause 39: The method of Clause 32, further comprising achieving a loss moduli similar to a loss moduli of patient-derived synovial fluid.

Clause 40: The method of Clause 32, further comprising forming a knee osteoarthritis simulated synovial fluid.

Clause 41: The method of Clause 32, the cytokines comprising one or more of basic fibroblast growth factor (bFGF), epidermal growth factor (EGF), eotaxin, granulocyte-colony stimulating factor (G-CSF), granulocyte-macrophage colony-stimulating factor (GM-CSF), hepatocyte growth factor (HGF), IFN-alpha, IFN-gamma, IL-2, IL-2R, IL-6, IL-7, IL-8, IL-12, IL-13, IL-15, IL-17A, interferon gamma-induced protein 10 (IP-10), monocyte chemoattractant protein-1 (MCP-1), monokine induced by interferon-gamma (MIG), macrophage inflammatory protein-1 alpha (MIP-1alpha), macrophage inflammatory protein-1 beta (MIP-1beta), regulated on activation-normal T cell expressed and secreted (RANTES), tumor necrosis factor alpha (TNF-alpha), or vascular endothelial growth factor (VEGF).

Clause 42: The method of Clause 41, the cytokines comprising bFGF, G-CSF, HGF, MIG, or MCP-1.

Clause 43: The method of Clause 32, wherein adding the high molecular weight hyaluronic acid comprises adding a higher concentration of the high molecular weight hyaluronic acid to the mixture compared to the low molecular weight hyaluronic acid.

Clause 44: The method of any of Clauses 32-43, wherein the high molecular weight hyaluronic acid comprises a molecular weight greater than 0.3 megadaltons (MDa).

Clause 45: The method of any of Clauses 32-44, wherein the low molecular weight hyaluronic acid comprises a molecular weight equal to or less than 0.3 megadaltons (MDa).

Clause 46: The method of Clause 32, wherein the predetermined time ranges from approximately 2 hours to approximately 72 hours.

Clause 47: The method of Clause 46, wherein the predetermined time comprises approximately 48 hours.

Clause 48: The method of any of Clauses 32-47, further comprising storing the mixture at a temperature below 0° C.

Clause 49: A method of analyzing one or more cellular responses from human cells, the method comprising: exposing the sample of human cells to a simulated synovial fluid comprising the composition of Clause 1; and performing a 2D synovial fluid potency assay on the human cells exposed to the simulated synovial fluid.

Clause 50: The method of Clause 49, further comprising performing a 3D synovial fluid exposure potency assay on the human cells exposed to the simulated synovial fluid.

Clause 51: The method of Clause 50, further comprising: exposing the sample of human cells to a patient-derived synovial fluid; performing a 2D synovial fluid potency assay on the human cells exposed to the patient-derived synovial fluid; and comparing the cellular responses for the human cells exposed to the simulated synovial fluid to the cellular responses of the human cells exposed to the patient-derived synovial fluid.

Clause 52: The method of Clause 51, further comprising performing a 3D synovial fluid exposure potency assay on the human cells exposed to the patient-derived synovial fluid.

Clause 53: The method of Clause 51, further comprising identifying one or more of: a change in cell proliferation; a presence of mammalian cells; a presence of bone marrow-derived mesenchymal stem cell (BM-MSC); or a presence of umbilical cord tissue-derived mesenchymal stem cells (UCT-MSCs).

Clause 54: A method of predicting a cellular response to a treatment for knee osteoarthritis, comprising: providing a simulated synovial fluid comprising the composition of any of Clauses 1-31; performing one or more cell therapies on a first sample of human cells; exposing the first sample of human cells to the simulated synovial fluid; and performing a 2D synovial fluid potency assay on the first sample of human cells exposed to the simulated synovial fluid.

Clause 55: The method of Clause 54, further comprising: performing a 3D synovial fluid exposure potency assay on the first sample of human cells exposed to the simulated synovial fluid.

Clause 56: The method of Clause 54 or 55, further comprising: providing a patient-derived synovial fluid; performing one or more cell therapies on a second sample of human cells; exposing the second sample of human cells to the patient-derived synovial fluid; and performing a 2D synovial fluid potency assay on the second sample of human cells exposed to the patient-derived synovial fluid.

Clause 57: The method of Clause 56, further comprising performing a 3D synovial fluid exposure potency assay on the second sample of human cells exposed to the patient-derived synovial fluid.

Clause 58: The method of Clause 56 or 57, further comprising comparing the cellular responses for the first sample of human cells to the cellular responses of the second sample of human cells, thereby predicting a cellular response to a treatment for knee osteoarthritis.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A composition comprising:
two or more cytokines, and
one or more of keratan sulfate, chondroitin sulfate, or hyaluronic acid;
wherein the composition simulates a fluid from a patient.

2. The composition of claim 1, wherein at a shear rate of approximately 0.1 reciprocal seconds (s⁻¹), the composition comprises a viscosity ranging from approximately 100 millipascal second (mPa· s) to approximately 10,000 mPa·s.

3. The composition of claim 1, wherein at an angular frequency of approximately 0.1 radians per second (rad/sec), the composition comprises a loss modulus ranging from approximately 0.01 pascal (Pa) to approximately 1 Pa.

4. The composition of claim 1, wherein at an angular frequency of approximately 0.1 radians per second (rad/sec), the composition comprises a storage modulus ranging from approximately 0.001 pascal (Pa) to approximately 1.5 Pa.

5. The composition of claim 1, the cytokines comprising one or more of basic fibroblast growth factor (bFGF), epidermal growth factor (EGF), eotaxin, granulocyte-colony stimulating factor (G-CSF), granulocyte-macrophage colony-stimulating factor (GM-CSF), hepatocyte growth factor (HGF), IFN-alpha, IFN-gamma, IL-2, IL-2R, IL-6, IL-7, IL-8, IL-12, IL-13, IL-15, IL-17A, interferon gamma-induced protein 10 (IP-10), monocyte chemoattractant protein-1 (MCP-1), monokine induced by interferon-gamma (MIG), macrophage inflammatory protein-1 alpha (MIP-1alpha), macrophage inflammatory protein-1 beta (MIP-1beta), regulated on activation-normal T cell expressed and secreted (RANTES), tumor necrosis factor alpha (TNF-alpha), or vascular endothelial growth factor (VEGF).

6. The composition of claim 5, the cytokines comprising bFGF, G-CSF, HGF, MIG, and MCP-1.

7. The composition of claim 6, wherein the bFGF comprises at least 40% based on a total weight per volume of cytokines in the composition.

8. The composition of claim 6, wherein the bFGF comprises from approximately 50 wt.% to approximately 60 wt.% based on the total weight per volume of the cytokines in the composition.

9. The composition of claim 1, further comprising human serum albumin (HSA).

10. The composition of claim 1, comprising both a high molecular weight hyaluronic acid and a low molecular weight hyaluronic acid.

11. The composition of claim 10, wherein the composition comprises:
high molecular weight hyaluronic acid present from approximately 5% to approximately 20%, based on a total weight per volume of the composition; and
low molecular weight hyaluronic acid present from approximately 0.01% to approximately 5%, based on a total weight per volume of the composition.

12. The composition of claim 11, wherein the high molecular weight hyaluronic acid is present in a higher concentration within the composition compared to the low molecular weight hyaluronic acid.

13. The composition of claim 1, wherein the simulated fluid has a viscosity similar to a viscosity of patient-derived synovial fluid.

14. The composition of claim 1, wherein the simulated fluid has a storage modulus similar to a storage modulus of patient-derived synovial fluid.

15. The composition of claim 1, wherein the simulated fluid has a loss modulus similar to a loss modulus of patient-derived synovial fluid.

16. The composition of claim 1, wherein the patient comprises a degenerative joint disease.

17. A method of making a composition for simulating a fluid from a patient, the method comprising:
creating a mixture comprising two or more cytokines and one or more of keratan sulfate, chondroitin sulfate, or human serum albumin;
adding a low molecular weight hyaluronic acid to the mixture;
adding a high molecular weight hyaluronic acid to the mixture; and
incubating the mixture for a predetermined time at a temperature ranging from approximately 0° C. to approximately 10° C.

18. The method of claim 17, further comprising:
obtaining one or more samples of patient-derived synovial fluid from one or more patients; and
analyzing a protein content of the one or more samples of patient-derived synovial fluid.

19. The method of claim 17, wherein the predetermined time ranges from approximately 2 hours to approximately 72 hours.

20. A method of analyzing one or more cellular responses from human cells, the method comprising:
exposing the sample of human cells to a simulated synovial fluid comprising the composition of claim 1; and
performing a 2D synovial fluid potency assay on the human cells exposed to the simulated synovial fluid.
